(19) United States
(12) Patent Application Publication
(10) Pub. No.: US 2008/0045595 A1
(43) Pub. Date: Feb. 21, 2008

(54) METHOD OF TREATING CANCER

(75) Inventors: Bruce Roth, Salt Lake City, UT (US); Vijay Baichwal, Salt Lake City, UT (US); Chris Pleiman, Holladay, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/769,322

(22) Filed: Jun. 27, 2007

Related U.S. Application Data
(63) Continuation of application No. PCT/US05/47313, filed on Dec. 27, 2005.
(60) Provisional application No. 60/639,372, filed on Dec. 27, 2004.

(51) Int. Cl. A61K 31/195; A61P 35/00

(52) U.S. Cl. 514/563

(57) ABSTRACT

The present invention provides therapeutic compounds and methods for the treatment of refractory and multidrug resistant cancers in patients in need of such treatment.

### RELATED PRIORITY APPLICATION

This application is a continuation of international application PCT/US2006/047313, filed Dec. 27, 2005, which claims benefit of U.S. Provisional Application Ser. No. 60/639,372 filed Dec. 27, 2004; the entire contents of which are incorporated herein by reference.

### FIELD OF THE INVENTION

The invention relates to therapeutic methods for the treatment of cancers, particularly refractory cancers that are resistant to certain antineoplastic agents.

### BACKGROUND OF THE INVENTION

Despite advances in surgery, radiation therapy, and antineoplastic treatments with antineoplastic agents, resistance of cancer cells, and thus cancers, to antineoplastic agents remains a major obstacle to treating specific cancers and improving cancer patients' prognoses. Resistance of cancers to antineoplastic agents can be observed either at the onset of treatment, when a patient fails to show a desired clinical response—in a phenomenon known as intrinsic resistance—or at a later time, when the disease recurs despite an initially successful response—a phenomenon known as acquired resistance. In either case, the cancer being treated is generally referred to as a “refractory” cancer. Along with severely compromising the efficacy of antineoplastic treatments with antineoplastic agents and the prognoses of treated patients, resistance to antineoplastic agents significantly limits the range of possibilities for subsequent treatments, since some cancer cells become resistant not only to the initial antineoplastic agent to which they have been exposed, but also to a variety of other antineoplastic agents, with different structures and mechanisms of action—a phenomenon referred to as multidrug resistance. See Pastan & Gottesman, Annu Rev Med. 42:277-286 (1991).

Drug resistance in cancer can arise from a variety of biochemical and molecular events, which ultimately result in the resistant tumor cells escaping death and continuing to divide and grow. Virtually any of these events, which can affect drug uptake, absorption, and metabolism, as well as drug efflux/influx and activation/inactivation, as well as DNA repair mechanisms—can potentially be targeted by antineoplastic agents to tip the balance in the cancer cell from tumor growth to apoptosis (Lee & MacGregor. Modern Drug Discovery. 7:45-49 (2004).

Multidrug resistance (MDR) is classically defined as resistance of cancer cells to the cytostatic or cytotoxic actions of multiple, structurally dissimilar and functionally divergent drugs commonly used in cancer chemotherapy (Gottesman. Cancer Res. 53:747-754 (1993)). Detailed studies of multidrug resistant cancers (MDR cancers) have suggested that the MDR phenomenon generally occurs at the level of the individual cells that comprise the MDR cancer. Cancer cells and cancers that exhibit MDR are said to be “refractory” to antineoplastic treatments.

MDR is a major cause of cancer chemotherapy failure in patients with refractory cancers. (See reviews by Gottesman. Cancer Res. 53:747-754 (1993); Gottesman. Annu Rev Med 53:615-627 (2002); and Kruh. Oncogene 22:7262-7264 (2003).) Although there are several distinct mechanisms by which cancer cells can develop drug resistance, MDR typically involves the ATP-dependent efflux of drugs from cells by a family of transmembrane proteins known as the ATP-binding cassette (ABC) transporters (Suzuki, et al. Curr Drug Metab. 2:367-377 (2001); Gottesman. Nat Rev Cancer 4:48-58 (2002); Gottesman. Annu Rev Med 53:615-627 (2002); Liscovitc & Lavie. IDrugs 5 (2002); and Kruh & Belinsky. Oncogene 22:7537-7552 (2003)). Frequently, one or more of the ABC transporters: ABCB1 (MDR-1, P glycoprotein, p-gp); ABCC1 (MRP1); and ABCG2 (BCRP, MXR) are over-expressed in the cells of drug resistant tumors, and thus, these proteins are implicated in the etiology of MDR (Szakacs, et al., Cancer Cell 6:129-137 (2004)).

In many instances of MDR cancers, the cells that are resistant to antineoplastic agents have been found to overexpress membrane transporters that mediate energy-dependent drug efflux of the antineoplastic agents. In such cancers the active transport of antineoplastic agents out of the cancer cell is classically the principle underlying reason for the resistance of the MDR cancer to the antineoplastic agents. In other instances of MDR cancers, cells of the MDR tumors have been found to overexpress other proteins that affect drug uptake, metabolism or mechanism of action, rather than drug efflux.

Such MDR to antineoplastic agents represents a significant challenge in the treatment of disseminated malignancies, as well as in the treatment of surgically inoperable tumors. Consequently, there is a clear need for new antineoplastic agents that circumvent, or otherwise escape, the mechanisms that underlie MDR—antineoplastic agents that are effective in killing MDR cancer cells and the refractory disseminated malignancies they cause.

More generally, cancers refractory to first line chemotherapy present significant challenges to the oncologist. Such drug resistant cancers generally have a poor prognosis and require sophisticated second line therapy that often involves combinations of antineoplastic agents. Better treatment of cancer patients with refractory cancers clearly requires new and effective antineoplastic agents.

### SUMMARY OF THE INVENTION

The inventors have discovered that 5-chloro-N-[2-(4-chloro-naphtalen-1-yloxy)-5-trifluoromethyl-phenyl]-2-hydroxy-benzamide is effective at killing cells from refractory cancer cell lines. The refractory cell lines found to be susceptible to 5-chloro-N-[2-(4-chloro-naphtalen-1-yloxy)-5-trifluoromethyl-phenyl]-2-hydroxy-benzamide are known to exhibit MDR. In particular, the inventors have discovered that 5-chloro-N-[2-(4-chloro-naphtalen-1-yloxy)-5-trifluoromethyl-phenyl]-2-hydroxy-benzamide is effective at killing cells from cancer cell lines that overexpress one or more of the ATP-binding cassette (ABC) transporters, including multidrug resistance protein 1 (MDR-1/ABCB1/P-glycoprotein), multidrug resistance associated protein 1 (MRP-1/ABCC1), and the breast cancer resistance protein (BCRP/MXR/ABCG2). Therefore, the present invention comprises therapeutic agents useful for the treatment of refractory cancers that are characterized by the overexpression of ABC transporters, including, at least, ABCB1, ABCC1 and/or ABCG2. The present invention also comprises methods of using 5-chloro-N-[2-(4-chloro-naphtalen-1-yloxy)-5-trifluoromethyl-phenyl]-2-hydroxy-benzamide, or a salt thereof, for the treatment of refractory cancers, particularly MDR cancers, as well as methods for using these therapeutic agents in combination with other antineoplastic agents for the treatment of such cancers. As such, the present invention provides methods for the treatment of cancers that are refractory to treatment by many “first line” antineoplastic regimens. The present invention also comprises the use of 5-chloro-N-[2-(4-chloro-naphtalen-1-yloxy)-5-trifluoromethyl-phenyl]-2-hydroxy-benzamide, and salts thereof, in the manufacture of medicaments for the treatment of patients with refractory cancers, as well as methods for identifying patients in need of such treatment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

Advantageously, the inventors have discovered that 5-chloro-N-[2-(4-chloro-naphtalen-1-yloxy)-5-trifluoromethyl-phenyl]-2-hydroxy-benzamide, the structure of which is provided by Formula I below, is effective at killing cells from cancer cell lines that exhibit MDR. Hence 5-chloro-N-[2-(4-chloro-naphtalen-1-yloxy)-5-trifluoromethyl-phenyl]-2-hydroxy-benzamide, or a salt thereof, can be used to treat patients with cancers that are refractory to first line antineoplastic treatments.

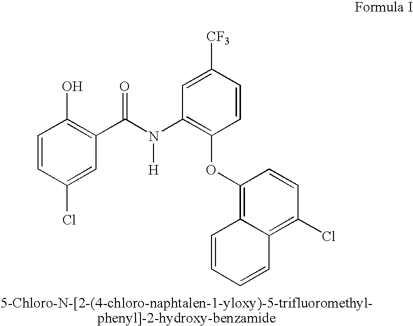

In particular, the inventors have discovered that 5-chloro-N-[2-(4-chloro-naphtalen-1-yloxy)-5-trifluoromethyl-phenyl]-2-hydroxy-benzamide is effective at killing cells from cancer cell lines that overexpress one of the ATP-binding cassette (ABC) transporter genes, including either multidrug resistance protein 1 (MDR-1/ABCB1/P-glycoprotein), multidrug resistance associated protein 1 (MRP-1/ABCC1) or the breast cancer resistance protein (BCRP/MXR/ABCG2) protein. Therefore, the present invention comprises therapeutic agents useful for the treatment of refractory cancers that are characterized by the overexpression of MDR-1, MRP-1 or BCRP, or any combination thereof. More generally, the present invention comprises therapeutic agents useful for the treatment of refractory cancers that are identified as a result of patients either not responding to a first line of treatment, or experiencing a relapse after initially responding to a first line of treatment. In other words, the present invention provides methods for second line (or subsequent) therapeutic treatments for cancers that are refractory to a first line (or subsequent) therapeutic treatment. Thus, the present invention comprises methods of using these therapeutic agents for the treatment of refractory cancers in patients in need of such treatment, and methods for identifying such patients.

The term “refractory cancers,” as used herein refers to any type of cancer that either fails to respond favorably to a first line of antineoplastic treatment, or alternatively, recurs or relapses after responding favorably to a first line of antineoplastic treatment. As such, refractory cancers include MDR cancers.

As mentioned above, MDR, with respect to cancer cells and cancers, is classically defined as resistance of tumor cells or tumors to the cytostatic or cytotoxic actions of multiple, structurally dissimilar and functionally divergent drugs commonly used in cancer chemotherapy (Gottesman. Cancer Res. 53:747-754 (1993)). Practically, MDR cancers are identified as cancers that either fail to respond, or, alternatively, exhibit a relapse or recurrence after initially responding to a first line of treatment with an antineoplastic agent. Such MDR cancers are also said to be refractory to the initial antineoplastic agent or antineoplastic treatment regimen, or refractory to the “first line” of antineoplastic agents or antineoplastic treatment.

Although there are many possible mechanisms whereby drug resistance can arise at the cellular level, most frequently MDR cancer cells overexpress membrane transporter proteins that mediate the energy-dependent efflux of the antineoplastic agents out of the cell. Particularly, in most MDR cancers, the cancer cells overexpress one or more of the ATP-binding cassette (ABC) transporters. Frequently, the ABC transporter protein being overexpressed is either the multidrug resistance protein 1 (MDR-1/ABCB1/P-glycoprotein), the multidrug resistance associated protein 1 (MRP-1/ABCC1), or the breast cancer resistance protein (BCRP/MXR/ABCG2) protein. In some MDR cancers, more than one ABC transporter is overexpressed in the MDR cancer cells.

Patients undergoing any antineoplastic treatment should be carefully monitored for signs of refractory cancer. This can be accomplished by monitoring the patient's cancer's response to the antineoplastic treatment. The response, lack of response, or relapse of the cancer to a first line of treatment can be determined by any suitable method practiced in the art.

For example, in the case of solid tumor cancers this can be accomplished by the assessment of tumor size and number. An increase in tumor size or, alternatively, tumor number, indicates that the tumor is not responding to the chemotherapy, or that a relapse has occurred. The National Cancer Institute of the National Institutes of Health has adopted and published guidelines for oncologists to use in assessing the response of tumors to cancer therapies. These “Response Evaluation Criteria in Solid Tumors,” or “RECIST” criteria are described in detail in Therasse et al, J. Natl. Cancer Inst. 92:205-216 (2000), and are summarized below.

With solid tumor cancers, tumor size and number can be assessed using an imaging technique. Examples of such techniques include magnetic resonance imaging (MRI), computed tomography (CT), spiral CT scans, and positron emission tomography (PET) scans, as well as a variety of other radiological techniques. Other techniques employed for assessing the response of solid tumors to therapeutic regimens include other non-invasive techniques such as ultrasound, or even manual palpitation.

In the case of hematological cancers, an increase in the number of cancer cells circulating in the bloodstream is indicative of the cancer not responding to the chemotherapy, or evidence that a relapse has occurred. In either case the cancer is said to be refractory to the initial chemotherapy. In such cases, evidence for increased numbers of circulating cancer cells can be assessed using a variety of histological and immunohistological techniques, including enzyme-linked immunosorbant assays (ELISAs), as well as various cell-sorting techniques.

For example, a patient with solid tumor cancer can be said to have refractory cancer when at least one of their tumors fails to respond to a first line of antineoplastic treatment and increases in volume by 10% or more over the course of the treatment, or shows any increase in metastasis as indicated by an increase in tumor numbers. Alternatively, if a patient's tumors respond to a first line of antineoplastic treatment, as indicated by a shrinkage of the tumor or complete cessation of tumor growth, but then show evidence of relapse in at least one tumor, by a subsequent increase in volume by 10% or more over the following four weeks, or an increase in tumor numbers, the patient can also be said to have refractory cancer.

In another example, a patient with hematological cancer can be said to have refractory cancer when their cancer fails to respond to a first line of antineoplastic treatment with an antineoplastic agent (or agents) and the number of cancerous cells in their blood increases by 5% or more over the course of the treatment, or the cancer shows evidence of metastasis as indicated by the formation of tumors. Alternatively, if a patient's hematological cancer responds to a first line of antineoplastic treatment, as indicated by a reduction in the number of cancerous cells in their blood, but then show evidence of relapse, by a subsequent 5% or greater increase in the number of cancerous cells in their blood over the following four weeks, the patient can also be said to have refractory cancer.

In those embodiments of the present invention involving the treatment of cancers characterized by the presence of solid tumors, a number of different criteria can be used to determine whether or not (a) the cancer being treated is a refractory cancer, and (b) the methods of the present invention are effective in treating the refractory cancer in question. Particularly, the “Response Evaluation Criteria in Solid Tumors,” or “RECIST” criteria, promulgated by the RECIST working group and adopted by the National Cancer Institute of the National Institutes of Health can be used. These criteria, which provide for a quantitative assessment of the efficacy of antineoplastic treatments, were described in an article entitled “New guidelines to evaluate the response to treatment in solid tumors” by Therasse and colleagues (Therasse, P., et al., J Natl Cancer Inst. 2000 Feb. 2;92(3):205-16), which is incorporated herein by reference in its entirety.

For the purpose of the present invention, patients with cancer characterized by the presence of solid tumors can, for example, be defined as having a refractory cancer when, after a first line of antineoplastic treatment, the patient is classified as having a “Stable Disease” or a “Progressive Disease,” using the RECIST criteria, at four weeks post-treatment. Additionally, patients with solid tumor cancers can, for example, also be defined as having refractory cancer when, four weeks after a first line of antineoplastic treatment the patient is classified using the RECIST criteria as having a “Complete Response” or a “Partial Response,” if there is a relapse of the cancer in the patient at any time after the four week evaluation period.

As an alternative to the RECIST criteria, the efficacy of the methods of the present invention can also be assessed by their effect on the survival, or average life expectancy, of the cancer patients being treated. A statistically significant increase in survival or average life expectancy of patients with refractory cancer being treated with the methods of the present invention indicates that the methods of the present invention are effective in treating refractory cancers.

The cytostatic or cytotoxic antineoplastic agents (drugs) most frequently associated with MDR are hydrophobic, amphipathic natural products, and their mimetics. Such antineoplastic agents associated with MDR include the Vinca alkaloids, anthracyclines, taxanes, including paclitaxel, epipodophyllotoxins, and the like). Therefore, patients being treated with such antineoplastic agents should be carefully monitored for signs of MDR cancer. This can be accomplished by monitoring the patient's cancer's response to antineoplastic treatment. The response, lack of response, or relapse of the cancer to a first line of treatment can be determined by any suitable method. In the case of solid tumor cancers this is typically accomplished by the assessment of tumor size and number. An increase in tumor size or, alternatively, tumor number, indicates that the tumor is not responding to the antineoplastic treatment, or that a relapse has occurred. In the case of hematological cancers, an increase in the number of cancer cells circulating in the bloodstream is indicative of the cancer not responding to the antineoplastic treatment, or evidence that a relapse has occurred. In either case the cancer is said to be refractory to the initial antineoplastic treatment.

With solid tumor cancers, tumor size and number can be assessed using an imaging technique. Such techniques include, e.g., magnetic resonance imaging (MRI), computed tomography (CT), spiral CT scans, and positron emission tomography (PET) scans, as well as a variety of other radiological techniques. They can also include other non-invasive techniques such as ultrasound, or even manual palpitation.

In the case of hematological cancers, evidence for increased numbers of circulating cancer cells can be assessed using a variety of histological and immunohistological techniques, including, but not limited to, enzyme-linked immunosorbant assays (ELISAs), as well as various cell-sorting techniques.

For example, a patient with solid tumor cancer can be said to have MDR cancer when at least one of their tumors fails to respond to at least two different antineoplastic agents and increases in volume by 10% or more over the course of the treatment, or shows any increase in metastasis as indicated by an increase in tumor numbers. Alternatively, if a patient's tumors respond to a first line of antineoplastic treatment, as indicated by a shrinkage of the tumors or complete cessation of tumor growth, but then show evidence of relapse in at least one tumor, by a subsequent increase in volume by 10% or more over the following four weeks, or an increase in tumor numbers, and then the recurrent cancer fails to respond to a second antineoplastic agent, the patient can also be said to have MDR cancer.

In another example, a patient with hematological cancer can be said to have MDR cancer when their cancer fails to respond to at least two different antineoplastic agents and the number of cancerous cells in their blood increases by 5% or more over the course of the treatment, or the cancer shows evidence of metastasis as indicated by the formation of tumors. Alternatively, if a patient's hematological cancer responds to a first line of antineoplastic treatment, as indicated by a reduction in the number of cancerous cells in their blood, but then show evidence of relapse, by a subsequent 5% or greater increase in the number of cancerous cells in their blood over the following four weeks, and the recurrent cancer fails to respond to a second antineoplastic agent, the patient can also be said to have MDR cancer.

Given that MDR is generally associated with the overexpression of ABC transporter protein genes—especially the genes encoding multidrug resistance protein 1 (MDR-1/ABCB1/P-glycoprotein), the multidrug resistance associated protein 1 (MRP-1/ABCC1), or the breast cancer resistance protein (BCRP/MXR/ABCG2) protein (See Porst et al., J. Natl. Cancer Inst. 92:1295-1302 (2000) and Gottesman et al., Nature Reviews Cancer 2:48-58 (2002))—a variety of assays have either been developed or envisioned to prognose and diagnose MDR cancers via the quantitation of the products of these genes. Such assays may assess the level of expression of the mRNAs encoding the ABC transporter proteins, or may assess the level of expression of the proteins themselves. Other assays that assess protein levels by assessing protein function have been developed or proposed. For assays that involve the direct quantitation of mRNA or expressed protein, the assay must be conducted on biopsy samples obtained from cancerous tissues or tumors, or on cells or extracts isolated from such biopsies.

Quantification of mRNAs encoding specific ABC transporter proteins can be conducted by any appropriate method known in the art. For example, quantification of mRNAs encoding specific ABC transporter proteins can be conducted quantitative polymerase chain reaction amplification assays using primers specific to the nucleic acids encoding the ABC transporter protein being studied, following reverse transcription of the mRNA (quantitative RT-PCR). Alternatively, the mRNAs encoding a number of ABC transporter proteins can be quantitated en mass, using expression profiling techniques and nucleic acid microarrays. Examples of such expression profiling techniques have recently been reported. For example, see Young, et al. Clin. Cancer Res. 7:1798-1804 (2001); Lee & MacGregor. Modern Drug Discovery 7:45-49 (July 2004); Szakacs et al. Cancer Cell 6:129-137 (2004); Annereau et al., Molec. Pharmacol. 66:1397-1405 (2004); Lee & MacGregor. Pharmacogenomics. 5:611-625 (2004); and Gillet et al., Cancer Res. 64:8987-8993 (2004), all of which are incorporated by reference herein in their entirety.

Direct quantification of expressed protein can be by any suitable technique, but generally involves the use of antibodies that specifically bind with the ABC transporter proteins. Examples of such techniques are provided in Scheffer et al., Cancer Research 60:5269-5277 (2000) and Young, et al. Clin. Cancer Res. 7:1798-1804 (2001), which are incorporated by reference herein in their entirety. Indirect quantification of expressed protein, as mentioned above, can be accomplished by assessing protein function. Examples of such methods have been described. For example, see Szakacs et al. Pathol Oncol Research 4:251-257 (1998), which is incorporated by reference herein in its entirety.

Therapeutic Compositions of the Present Invention:

The present invention also provides for the preparation and administration of a therapeutic compositions designed to deliver a therapeutically effective amount of 5-chloro-N-[2-(4-chloro-naphtalen-1-yloxy)-5-trifluoromethyl-phenyl]-2-hydroxy-benzamide, or a salt or prodrug thereof, to a patient.

The compound, 5-chloro-N-[2-(4-chloro-naphtalen-1-yloxy)-5-trifluoromethyl-phenyl]-2-hydroxy-benzamide, is disclosed in PCT publication WO 2004/006858, which also provides methods for synthesizing the compound. The compound, or pharmaceutical salts thereof, can be administered in any suitable composition by any suitable route, as disclosed in PCT application PCT/US05/43481.

5-chloro-N-[2-(4-chloro-naphtalen-1-yloxy)-5-trifluoromethyl-phenyl]-2-hydroxy-benzamide has been discovered to exhibit low bioavailability when administered orally. Accordingly, in one aspect of the invention, liquid pharmaceutical compositions are provided that allow for parenteral administration of compounds of the invention.

The amount of compound per unit volume of pharmaceutical composition may vary. For example, the amount of compound in the pharmaceutical composition may be at least about 0.01 mg/ml or at least about 1 mg/ml. In another example, the amount of compound in the pharmaceutical composition is between about 1 mg/ml and about 50 mg/ml. In a specific example, the amount of compound in the pharmaceutical composition may be between about 5 mg/ml and about 15 mg/ml.

In one embodiment, a pharmaceutical composition is provided wherein the liquid vehicle is aqueous and comprises an aqueous diluent. Pharmaceutically acceptable aqueous diluents include solutions commonly used to prepare substances for parenteral administration, such as intravenous administration. Exemplary aqueous diluents include water, saline, and aqueous dextrose solutions, such as 5% dextrose in water (D5W).

Representative concentrations of 5-chloro-N-[2-(4-chloro-naphtalen-1-yloxy)-5-trifluoromethyl-phenyl]-2-hydroxy-benzamide in aqueous diluents, are expressed as μg or mg of compound per mL aqueous diluent, and include concentrations from about 1 μg/mL to about 50 mg/mL, and from about 1 mg/mL to about 10 mg/mL.

In other embodiments, a pharmaceutical composition is provided wherein the liquid vehicle comprises one or more non-ionic surfactants. As used herein, the term “surfactant” refers to an agent that can solubilize compounds of the invention, and maintain solubilization once diluted into aqueous solutions. Exemplary surfactants are capable of completely solubilizing, or at least partially solubilizing the compounds of the invention and may form micelles or other self-associated structures when introduced into an aqueous environment.

Although surfactants can be anionic, cationic, amphoteric or non-ionic, in exemplary embodiments of the present invention the surfactants are non-ionic. Pharmaceutically acceptable non-ionic surfactants typically include esters and ethers of polyoxyalkylene glycols, esters and ethers of polyhydric alcohols, or esters and ethers of phenols. Poloxamers and poloxamines are also examples of non-ionic surfactants. Specific examples of non-ionic surfactants include, but are not limited to, polyoxyethylene castor oil derivatives.

Beneficially, surfactants used in embodiments of the invention may allow the 5-chloro-N-[2-(4-chloro-naphtalen-1-yloxy)-5-trifluoromethyl-phenyl]-2-hydroxy-benzamide, and salts thereof, to be solubilized in, and delivered by way of, pharmaceutically acceptable aqueous diluents. Such final formulations with substantially stable micelles in an aqueous diluent may be delivered by parenteral routes, especially via intravenous injection and infusion.

The amount of surfactant per unit volume of pharmaceutical composition may vary. For example, a surfactant may makeup about 20 wt % to about 99.9 wt % of the pharmaceutical composition with the remainder made up of excipients, drug, stabilizing agents and the like. Exemplary ratios (weight/volume, i.e., weight of compound/volume of pharmaceutical composition) between compounds and the pharmaceutical composition may be from about 0.001 g/L to about 500 g/L, from about 1.0 g/L to about 300 g/L, and from about 5 g/L to about 100 g/L. In a specific embodiment, the non-ionic surfactant is polyoxyl 35 castor oil wherein the weight to weight ratio between 5-chloro-N-[2-(4-chloro-naphtalen-1-yloxy)-5-trifluoromethyl-phenyl]-2-hydroxy-benzamide, or a salt thereof, and the surfactant is from about 1:200 to about 1:20.

A pharmaceutical composition of the present invention having a non-aqueous liquid vehicle comprising a surfactant optionally may also include one or more viscosity reducing agents. For example, a liquid vehicle comprising one ore more non-ionic surfactants may also contain one or more viscosity reducing agents.

As used herein, the term “viscosity reducing agent” means a pharmaceutically acceptable compound that, when mixed with a surfactant reduces the viscosity of the surfactant or liquid vehicle to such an extent that the resulting solution can be readily handled by syringes and can be readily sterile filtered. Advantageously, viscosity reducing agents of the instant invention reduce the viscosity of the surfactant having 5-chloro-N-[2-(4-chloro-naphtalen-1-yloxy)-5-trifluoromethyl-phenyl]-2-hydroxy-benzamide, or a salt thereof, dissolved therein to the point where the resulting solution can be filtered through sterile filters common to the art of sterile filtration pharmaceutical manufacturing processes, and that are often described as filters bearing pores of 0.22 micrometers (μm), or less at room temperature. Such viscosity reducing agents allow for the use of surfactants that, by themselves, are too viscous to be readily handled by syringes or sterile filtered, in the compositions and formulations of the instant invention.

In particular embodiments, a viscosity reducing agent may range from about 5 wt % to about 80 wt % of the pharmaceutical composition. In certain embodiments, the viscosity reducing agent is about 20 wt % to about 60 wt % of the composition. In other embodiments a viscosity reducing agent accounts for up to about 50 wt % of the composition.

As is true of each of the other constituents of the compositions of the present invention, the precise amount of viscosity reducing agent included in the pharmaceutical composition of the present invention may be varied. For example, the amount of viscosity reducing agent may vary to achieve a sought benefit in the syringability or filterability of the pharmaceutical composition.

Representative viscosity reducing agents include C₁₋₅ alkanol, benzyl alcohol, monoesters of glycerol, and aliphatic mono carboxylic acids. In a specific embodiment, the viscosity reducing agent is ethanol. In another specific embodiment, the ratio of non-ionic surfactant to viscosity reducing agent is from about 10:1 to about 1:10 (v/v). In a specific embodiment, the non-aqueous liquid vehicle comprising one or more non-ionic surfactants further includes an aqueous diluent, such as water, saline, and/or aqueous dextrose solution. For example, the ratio of non-ionic surfactant to aqueous diluent may be at least about 1:1 (v/v), such as from about 100:1 to about 1:100 (v/v).

A pharmaceutical composition of the present invention may optionally include excipients. For example, a liquid vehicle comprising a non-ionic surfactant may also contain one or more excipients.

Exemplary excipients include such agents as preservatives, antioxidants, pH adjusting agents, osmolarity adjusting agents, and stabilizers. Preservatives are generally viewed as agents that prevent or inhibit microbial growth in a formulation. Representative preservatives include parabens (e.g. methyl, ethyl, propyl, and butyl paraben), ethanol, isopropanol, sodium benzoate, benzyl alcohol, chlorobutanol, phenol, potassium sorbate, thimerosal, and benzalkonium chloride.

Antioxidants generally serve to protect the components of the compositions from oxidative damage. Examples of antioxidants include ascorbic acid, sodium ascorbate, ascorbyl palmitate, BHA (butylated hydroxyanisole) BHT (butylated hydroxytoluene), vitamin E, vitamin E PEG 1000, and TPGS.

Excipients may also be pharmaceutically acceptable pH adjusting agents and/or osmolarity adjusting agents. Such agents are used to improve the characteristics of the pharmaceutical composition so that it can be used to prepare solutions and liquids that are suitable for parenteral administration, especially intravenous injection and infusion. Suitable pH adjusting agents include buffers (e.g., phosphate, acetate, carbonate, tromethamine, citrate, lactate), acidifying agents (e.g., hydrochloric acid, tartaric acid, acetic acid, citric acid), and alkalizing agents (such as sodium or potassium hydroxide, monoethanolamine, diethanolamine, triethanolamine). Examples of suitable osmolarity adjusting agents include any pharmaceutically acceptable water soluble compound, either ionic or nonionic in nature, such as glucose, sucrose, fructose, sodium chloride, sodium lactate, sorbitol, mannitol, glycerin, polyethylene glycols 400 to 4000, and pharmaceutical buffer salts.

In specific embodiments, the liquid vehicle comprises at least one non-ionic surfactant and at least one antioxidant, such as BHT (butylated hydroxytoluene). In certain embodiments, the liquid vehicle comprises at least one non-ionic surfactant and at least one aqueous diluent, such as water, saline, and/or aqueous dextrose solution. For example, the ratio of non-ionic surfactant to aqueous diluent may be at least about 1:1 (v/v), such as from about 1:2 to about 1:100 (v/v).

The pharmaceutical compositions of the invention may also be a combination of one or more surfactants, viscosity reducing agents, excipients, and aqueous diluents. In a specific embodiment, the invention provides a pharmaceutical composition suitable for parenteral administration to a mammal, comprising a therapeutically effective amount of 5-chloro-N-[2-(4-chloro-naphtalen-1-yloxy)-5-trifluoromethyl-phenyl]-2-hydroxy-benzamide, or a salt thereof, in admixture with a liquid vehicle comprising a non-ionic surfactant, a viscosity reducing agent, and an aqueous diluent selected from water, saline, and aqueous dextrose solution, wherein the weight ratio between said compound and said non-ionic surfactant is from about 1:200 to about 1:20, the weight ratio between said non-ionic surfactant and said viscosity reducing agent is from about 1:10 to about 10: 1, and the v:v ratio between said non-ionic surfactant and said aqueous diluent is from about 100:1 to about 1:100.

Thus, an important aspect of the present invention is the application of the methods of the present invention to treat cancer patients having tumors that exhibit resistance to other antineoplastic agents. In one embodiment, a pharmaceutical composition comprising a therapeutically effective amount of 5-chloro-N-[2-(4-chloro-naphtalen-1-yloxy)-5-trifluoromethyl-phenyl]-2-hydroxy-benzamide, or a salt thereof, is administered to a cancer patient who has previously been treated with one or more antineoplastic agents or anticancer drugs, and whose cancer was found to be non-responsive or refractory to the previous treatment.

In another embodiment, a pharmaceutical composition comprising a therapeutically effective amount of 5-chloro-N-[2-(4-chloro-naphtalen-1-yloxy)-5-trifluoromethyl-phenyl]-2-hydroxy-benzamide, or a salt thereof, is administered to a patient who has previously been treated with another antineoplastic agent, and whose cancer was found to have developed resistance to the previously administered antineoplastic agent.

In still another embodiment, a pharmaceutical composition comprising a therapeutically effective amount of 5-chloro-N-[2-(4-chloro-naphtalen-1-yloxy)-5-trifluoromethyl-phenyl]-2-hydroxy-benzamide, or a salt thereof, is administered to a patient who has been previously treated with another antineoplastic agent and whose cancer is found to be refractory to that other antineoplastic agent.

In yet still another embodiment, a pharmaceutical composition comprising a therapeutically effective amount of 5-chloro-N-[2-(4-chloro-naphtalen-1-yloxy)-5-trifluoromethyl-phenyl]-2-hydroxy-benzamide, or a salt thereof, is administered to a cancer patient who has previously been treated with another antineoplastic agent, and whose cancer was initially responsive to the previously administered antineoplastic agent, but was subsequently found to have relapsed.

Another important aspect of the present invention is the application of the methods of the present invention to treat cells isolated from tumors of cancer patients that exhibit resistance to other antineoplastic agents or anticancer drugs.

Thus, the above methods of the present invention can be practiced by or comprise treating cells in vitro or within a warm-blood animal, particularly mammal, more particularly a human, with a therapeutically effective amount of 5-chloro-N-[2-(4-chloro-naphtalen-1-yloxy)-5-trifluoromethyl-phenyl]-2-hydroxy-benzamide, or a salt thereof, according to the present invention. As used herein, the phrase “treating . . . with . . . 5-chloro-N-[2-(4-chloro-naphtalen-1-yloxy)-5-trifluoromethyl-phenyl]-2-hydroxy-benzamide” means either administering 5-chloro-N-[2-(4-chloro-naphtalen-1-yloxy)-5-trifluoromethyl-phenyl]-2-hydroxy-benzamide to cells, or to an animal, directly, or administering to cells or to an animal another agent that causes or results in the formation of 5-chloro-N-[2-(4-chloro-naphtalen-1-yloxy)-5-trifluoromethyl-phenyl]-2-hydroxy-benzamide inside the cells or the animal.

In one embodiment, the methods of the present invention comprise contacting cells in vitro, or within a warm-blood animal, particularly mammal, more particularly a human, with a therapeutically effective amount of 5-chloro-N-[2-(4-chloro-naphtalen-1-yloxy)-5-trifluoromethyl-phenyl]-2-hydroxy-benzamide, or a salt thereof. The term “contacting,” as used herein refers to any suitable delivery method for bringing 5-chloro-N-[2-(4-chloro-naphtalen-1-yloxy)-5-trifluoromethyl-phenyl]-2-hydroxy-benzamide, or a salt thereof, in contact with the cancer cells that are either refractory to treatment with another antineoplastic agent, or have exhibited MDR. For applications in vitro, this embodiment encompasses merely adding 5-chloro-N-[2-(4-chloro-naphtalen-1-yloxy)-5-trifluoromethyl-phenyl]-2-hydroxy-benzamide, or a salt thereof, solubilized in a liquid vehicle to the cell culture medium.

For applications in vivo, 5-chloro-N-[2-(4-chloro-naphtalen-1-yloxy)-5-trifluoromethyl-phenyl]-2-hydroxy-benzamide, or a salt thereof, can be delivered to the refractory or MDR cancer cells using any suitable method known to those of ordinary skill in the art of drug delivery, such as, e.g., those described in PCT publication WO 2004/006858. However, parenteral delivery, and especially intravenous delivery, is preferred, and those skilled in the art of drug delivery are familiar with the various apparati designed for drug delivery via this route of administration. Hence, pharmaceutical compositions for the parenteral delivery, and especially intravenous delivery, of 5-chloro-N-[2-(4-chloro-naphtalen-1-yloxy)-5-trifluoromethyl-phenyl]-2-hydroxy-benzamide, or a salt thereof, have been described herein and in PCT application PCT/US05/43481.

When contacting cells in culture, effective doses of 5-chloro-N-[2-(4-chloro-naphtalen-1-yloxy)-5-trifluoromethyl-phenyl]-2-hydroxy-benzamide, or salts thereof, are typically those close to the IC₅₀ (see Examples 1, 2, & 3, below). For in vivo applications, acute toxic doses are determined in pre-clinical trials by treating animals at fractions or multiples of the IC₅₀ and assessing toxicity. Preferred dosages and dosing regimens for humans will be perfected following clinical trials using methods well known to those of ordinary skill in the art of clinical trials, and will be further optimized for particular types of refractory or MDR cancers. Expected dosages for the treatment of patients with refractory or MDR cancers are preferably from about 5 mg 5-chloro-N-[2-(4-chloro-naphtalen-1-yloxy)-5-trifluoromethyl-phenyl]-2-hydroxy-benzamide, or a salt thereof, per kg of body weight to about 250 mg/kg, and more preferably from about 5 mg/kg to about 100 mg/kg.

The methods of the present invention may also include a step of diagnosing a patient, and determining whether they have an MDR cancer, by determining, either directly or indirectly, whether the cells of their cancer overexpress one or more of the ABC transport proteins, including multidrug resistance protein 1 (MDR-1/ABCB 1/P-glycoprotein), multidrug resistance associated protein 1 (MRP-1/ABCC1), and the breast cancer resistance protein (BCRP/MXR/ABCG2) protein. For this aspect of the present invention a diagnostic assay is conducted, usually on a sample obtained from the patient, in order to assess the level of expression of one or more ABC transporter proteins, or the mRNAs that encode them. The results of this diagnostic assay are then compared with established “baseline” values in order to determine whether the cancer cells of the patient are expressing elevated levels of one or more ABC transporter proteins, or the mRNAs that encode them. Elevated levels of expression, either of the ABC transporter proteins themselves, or of the mRNAs that encode them, are indicative of MDR cancer, and suggest that the methods and compositions of the present invention would be particularly useful in treating the patient from whom the sample was obtained.

As noted previously, the diagnostic assays used for this aspect of the present invention, can either assess the level of expression of one or more ABC transporter proteins themselves, or, alternatively, can assess the level of expression of the mRNAs that encode them. Such diagnostic assays are known in the art and can advantageously be employed as part of the methods of the instant invention. Assays designed to assess the level of expression of one or more ABC transporter proteins themselves generally utilize antibodies that selectively bind an epitope on one of the ABC transporter proteins. Such assays can include Western blots enzyme-linked immunosorbent assays (ELISA) and enzyme-linked immunofiltration assays (ELIFA). Assays suitable for this purpose have been disclosed in Scheffer et al., Cancer Research 60:5269-5277 (2000) and Young, et al. Clin. Cancer Res. 7:1798-1804 (2001), which are incorporated by reference herein in their entirety.

Assays designed to assess the level of expression of mRNAs encoding one or more ABC transporter proteins can also be used and generally involve selective nucleic acid amplification or hybridization technologies. Amplification-based assays include, for example quantitative polymerase chain reaction (PCR)-based assays, such as real time PCR. Hybridization-based assays include semi-quantitative Southern blots and, preferably, microarray-based quantification of cDNAs. The latter technique is preferred because it allows for the simultaneous assessment of mRNA expression levels for multiple mRNAs, such as those encoding different ABC transporter proteins. Assays suitable for this purpose have been disclosed in Young, et al Clin. Cancer Res. 7:1798-1804 (2001); Lee & MacGregor. Modern Drug Discovery 7:45-49 (July 2004); Szakacs et al. Cancer Cell 6:129-137 (2004); Annereau et al., Molec. Pharmacol. 66:1397-1405 (2004); Lee & MacGregor. Pharmacogenomics. 5:611-625 (2004); and Gillet et al., Cancer Res. 64:8987-8993 (2004), all of which are incorporated by reference herein in their entirety.

Through the use of assays designed to assess the level of expression of the ABC transporter proteins, or the mRNAs that encode them, and samples of cancer cells obtained from patients, one of skill in the art can readily assess whether or not the patient likely has an MDR cancer, since it is known in the art that the cells of many MDR cancers overexpress the ABC transporter proteins. In identifying patients having MDR cancers in this manner, the skilled artisan so identifies patients that would likely benefit from the therapeutic methods and pharmaceutical compositions of the present invention.

Consequently, in another aspect of the present invention methods are provided for treating a patient with an MDR cancer by first determining, from a sample taken from the patient, whether the cancer cells in that sample overexpress an ABC transporter protein, and if so, treating that patient with a therapeutically effective amount of 5-chloro-N-[2-(4-chloro-naphtalen-1-yloxy)-5-trifluoromethyl-phenyl]-2-hydroxy-benzamide, or a salt thereof. Any of the assays described above, whether based upon the detection of the ABC transporter proteins themselves, or the nucleic acids that encode them, can be employed in the practice of this aspect of the invention, as well as any other assay that can detect the overexpression of ABC transporter proteins by cancer cells.

The present invention also includes therapeutic methods comprising administering to cells in vitro, or within a warm-blooded animal, an effective amount of 5-chloro-N-[2-(4-chloro-naphtalen-1-yloxy)-5-trifluoromethyl-phenyl]-2-hydroxy-benzamide, or a pharmaceutically acceptable salt or prodrug of 5-chloro-N-[2-(4-chloro-naphtalen-1-yloxy)-5-trifluoromethyl-phenyl]-2-hydroxy-benzamide, wherein said therapeutic method is useful to treat MDR cancer. Such diseases include, but are not limited to: Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In practicing the therapeutic methods of the present invention, effective amounts of pharmaceutical compositions containing therapeutically effective concentrations of 5-chloro-N-[2-(4-chloro-naphtalen-1-yloxy)-5-trifluoromethyl-phenyl]-2-hydroxy-benzamide, or a salt thereof, are formulated for oral, parenteral, local and topical application, for the treatment of MDR neoplastic diseases and other MDR disorders, are administered to an individual exhibiting the symptoms of one or more of these disorders. An effective amount of 5-chloro-N-[2-(4-chloro-naphtalen-1-yloxy)-5-trifluoromethyl-phenyl]-2-hydroxy-benzamide, or a salt thereof, for treating a particular disease or disorder is an amount that is sufficient to ameliorate, or in some manner reduce, the symptoms associated with the disease or disorder. Such amount may be administered as a single dosage or may be administered according to a treatment regimen, whereby it is effective to ameliorate, or in some manner reduce, the symptoms associated with the disease or disorder. The amount administered may serve to cure the disease but, more typically, it is an amount necessary to ameliorate the symptoms of the disease. Typically, repeated administration is required in order to achieve the desired amelioration of symptoms and/or cure of the disease or disorder.

In one aspect of the therapeutic methods of the present invention, 5-chloro-N-[2-(4-chloro-naphtalen-1-yloxy)-5-trifluoromethyl-phenyl]-2-hydroxy-benzamide, or a salt thereof, is used to treat MDR cancers in warm-blooded animals previously treated with an anti-cancer compound that is not 5-chloro-N-[2-(4-chloro-naphtalen-1-yloxy)-5-trifluoromethyl-phenyl]-2-hydroxy-benzamide, or a salt thereof. In this aspect of the present invention, 5-chloro-N-[2-(4-chloro-naphtalen-1-yloxy)-5-trifluoromethyl-phenyl]-2-hydroxy-benzamide, or a salt thereof, is used in a so-called “second line” treatment of an MDR cancer, wherein the previously administered anti-cancer compound was administered during a “first line” of treatment. Importantly, 5-chloro-N-[2-(4-chloro-naphtalen-1-yloxy)-5-trifluoromethyl-phenyl]-2-hydroxy-benzamide, or a salt thereof, can also be administered as a third—or subsequent—line treatment, when two or more other anti-cancer compounds have been administered to the same patient.

In one embodiment of the present invention, the anti-cancer compound used in the initial treatment regimen(s) can be an alkylating agent, an antimitotic agent, a tubulin inhibitor, a topoisomerase I inhibitor, a topoisomerase II inhibitor, an RNA/DNA antimetabolite, a DNA antimetabolite, an EGFR inhibitor, an angiogenesis inhibitor, or a proteosome inhibitors, or some combination thereof. In another embodiment of the present invention, the anti-cancer compound used in the initial treatment regimen(s) can be an anthracylcline, a vinca-akyloid, a taxane, or a metal-complex, or some combination thereof. In yet another embodiment of the present invention, the anti-cancer compound used in the initial treatment regimen(s) can be actinomycin-D, bleomycin, bisantrene, aclarubicin, doxorubicin, daunorubicin, epirubicin, idarubicin, docetaxel, paclitaxel, etoposide, teniposide, topotecan, mitoxantrone, vinblastine, vincristine, vinorelbine, homoharringtonine, cisplatin, chlorambucil, melphalan, cyclophosamide, ifosfamide, mitoguazone, elliptinium, fludarabine, octreotide, retinoic acid, tamoxifen, Gleevec® (imatinib mesylate), alanosine or SN-38, or some combination thereof.

The methods of the present invention can be used to treat any neoplastic disease or disorder wherein the cells or tumors associated with the neoplastic disease or disorder exhibit multidrug resistance. Thus, in one set of embodiments, the methods of the present invention can be used to treat refractory cancers characterized by the presence of solid tumors. Importantly, in these embodiments, the methods of the present invention can be used to treat metastatic cancers, including, but not limited to, cancers of the skin, colon, rectum, esophagus, thyroid, liver, pancreas, kidney, bladder, lung, brain, breast, ovary, testicles or prostate. In another set of embodiments, the methods of the present invention can be used to treat refractory hematological cancers, including, but not limited to, leukemias, myelomas, non-Hodgekin's lymphomas or Hodgekin's lymphomas. In still another set of embodiments, the methods of the present invention can be used to treat non-cancerous neoplastic diseases or disorders.

Therefore, the methods of the present invention can be applicable to a variety of tumors, i.e., abnormal growth, whether cancerous (malignant) or noncancerous (benign), and whether primary tumors or secondary tumors. Such disorders include but are not limited to lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart tumors such as myxoma, fibromas and rhabdomyomas; bone tumors such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain tumors such as gliomas (e.g., glioblastoma multiforme), anaplastic astrocytomas, astrocytomas, and oligodendrogliomas, medulloblastomas, chordoma, Schwannomas, ependymomas, meningiomas, pituitary adenoma, pinealoma, osteomas, and hemangioblastomas, craniopharyngiomas, chordomas, germinomas, teratomas, dermoid cysts, and angiomas; various oral cancers; tumors in digestive system such as leiomyoma, epidermoid carcinoma, adenocarcinoma, leiomyosarcoma, stomach adenocarcinomas, intestinal lipomas, intestinal neurofibromas, intestinal fibromas, polyps in large intestine, familial polyposis such as Gardner's syndrome and Peutz-Jeghers syndrome, colorectal cancers (including colon cancer and rectal cancer); liver cancers such as hepatocellular adenomas, hemangioma, hepatocellular carcinoma, fibrolamellar carcinoma, cholangiocarcinoma, hepatoblastoma, and angiosarcoma; kidney tumors such as kidney adenocarcinoma, renal cell carcinoma, hypernephroma, and transitional cell carcinoma of the renal pelvis; bladder cancers; cancers in the blood system including acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, mycosis fungoides, and myeloproliferative disorders (including myeloproliferative disorders are polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterus cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; cancerous or noncancerous growths of the pancreas; etc.

In addition, the methods are potentially also applicable to premalignant conditions to prevent, stop or slow the progression of such conditions towards malignancy, or cause regression of the premalignant conditions. Examples of premalignant conditions include hyperplasia, dysplasia, and metaplasia.

Thus, the term “treating cancer” as used herein, specifically refers to administering therapeutic agents to a patient diagnosed with refractory cancer, and preferably MDR cancer, i.e., having established the existence of refractory or MDR cancer in the patient, to inhibit the further growth or spread of the malignant cells in the cancerous tissue, and/or to cause the death of the malignant cells. The term “treating cancer” also encompasses treating a patient having premalignant conditions to stop the progression of, or cause regression of, the premalignant conditions.

The methods of the present invention may also be useful in treating or preventing other diseases and disorders caused by abnormal cell proliferation (hyperproliferation or dysproliferation), e.g., keloid, liver cirrhosis, psoriasis, etc. In addition, the methods may also find applications in promoting wound healing, and other cell and tissue growth-related conditions.

### EXAMPLE 1

### Cytotoxicity of 5-chloro-N-[2-(4-chloro-naphtalen-1-yloxy)-5-trifluoromethyl-phenyl]-2-hydroxy-benzamide in multidrug resistant cells overexpressing MDR-1

Abstract

Objective: This study was conducted to investigate whether cells over-expressing the P-glycoprotein pump (MDR-1, ABCB1) are sensitive to cytotoxic activity of 5-chloro-N-[2-(4-chloro-naphtalen-1-yloxy)-5-trifluoromethyl-phenyl]-2-hydroxy-benzamide in cell culture.

Methods: The tumor cells shown in Table 1 were plated at 5,000 cells/well in a 96 well tissue culture plate and grown for 24 hours at 37° C. The cells were then exposed to various concentrations of 5-chloro-N-[2-(4-chloro-naphtalen-1-yloxy)-5-trifluoromethyl-phenyl]-2-hydroxy-benzamide, Vinblastine or Taxotere® for 72 hours and the inhibition of cell growth due to exposure to the drug was determined using the ATPLite assay. Data are expressed as the concentration that inhibited growth by 50%.

Results: The in vitro activity of 5-chloro-N-[2-(4-chloro-naphtalen-1-yloxy)-5-trifluoromethyl-phenyl]-2-hydroxy-benzamide and Vinblastine and Taxotere®, which are approved anticancer drugs, is compared in tumor cells and drug resistant tumor cells in Table 1 below.

Conclusions: 5-chloro-N-[2-(4-chloro-naphtalen-1-yloxy)-5-trifluoromethyl-phenyl]-2-hydroxy-benzamide is equally active in tumor cells and cells over-expressing the MDR-1 protein, which are resistant to currently marketed anti-cancer compounds.

Introduction

Over-expression of the product of the MDR-1 (ABCB1) gene, the P-glycoprotein pump, confers resistance to a wide variety of agents including anthracyclines, vinca alkaloids and taxane derivatives. Cell lines with elevated expression of MDR-1 are less sensitive to the effect of these cytotoxic agents in vitro.

The objective of this study was to determine whether cell lines over-expressing the P-glycoprotein pump are sensitive to the cytotoxic activity of 5-chloro-N-[2-(4-chloro-naphtalen-1-yloxy)-5-trifluoromethyl-phenyl]-2-hydroxy-benzamide in cell culture.

Experimental Methods

Test/Control Articles

A. 5-chloro-N-[2-(4-chloro-naphtalen-1-yloxy)-5-trifluoromethyl-phenyl]-2-hydroxy-benzamide (Myriad Pharmaceuticals, Salt Lake City, Utah)
B. Vinblastine (Sigma Chemical Co.; St. Louis, Mo.)
C. Taxotere® (Aventis)
D. ATPLite® Assay Kit (PerkinElmer, Boston, Mass.)
   Materials

Stock solutions of compounds dissolved in DMSO were used to prepare compound dilutions for activity determination. 5-chloro-N-[2-(4-chloro-naphtalen-1-yloxy)-5-trifluoromethyl-phenyl]-2-hydroxy-benzamide was tested at a final concentration of 100 μM, 33.3 μM, 11.1 μM, 3.7 μM, 1.23 μM, 0.4 μM and 0.13 μM for both cell lines. Vinblastine and Taxotere® were tested at final concentrations of 100 nM, 33.3 nM, 11.1 nM, 3.7 nM, 1.23 nM, 0.4 nM and 0.13 nM for MCF-7 cells and at 10 μM 3.3 μM, 1.1 μM, 0.37 μM, 0.12 μM, 0.04 μM and 0.013 μM for NCl/ADR-RES cells. The final concentration of DMSO in all cases was adjusted to 0.2% v/v.

Test System

A. MCF-7, a human breast cancer cell line was obtained from ATCC (Catalog number: HTB-22)

B. NCl/ADR-RES, a multi drug resistant cell line over-expressing MDR-1 was obtained from NCl, Frederick, Md. This cell line has previously been called MCF-7/ADR by NCl.

All cells were cultured in RPMI-1640 supplemented with 10% fetal bovine serum, 2 mM Glutamax, 1 mM sodium pyruvate, 0.1 mM non-essential amino acids and 10 mM Hepes. MCF-7 cells were supplemented additionally with 10 μg/ml recombinant human insulin. All cells were grown at 37° C. in a humidified 5% CO₂ atmosphere.

Experimental

For testing the effect of compounds on cell growth, exponentially growing cells were harvested by trypsinization and plated in fresh growth medium at 50,000 cells/ml, one hundred microlitres cell suspension per well, in a 96-well flat-bottomed microtiter plate (Corning, Costar 3595). Twenty-four hours later, culture medium was replaced with fresh growth medium and varying concentrations of test compound.

Cells were incubated with compound for three days and cellular viability was determined at the end of the incubation by measuring intracellular ATP with ATPLite assay system. All compounds were tested triplicate.

Data Processing

Effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC₅₀). Data was analyzed by Prism software (GraphPad; San Diego, Calif.) by fitting it to a sigmoidal dose response curve. The IC₅₀ values obtained for individual data sets were combined to obtain a mean IC₅₀ and standard deviation of the mean.

Results

5-chloro-N-[2-(4-chloro-naphtalen-1-yloxy)-5-trifluoromethyl-phenyl]-2-hydroxy-benzamide showed similar cytotoxic activity against the normal and multi-drug resistant cell line with an IC₅₀ of 739 and 754 nM for MCF-7 and NCl/ADR-RES cells respectively (Table 1).

Vinblastine was less active against the MDR-1 over-expressing cells NCl/ADR-RES with an IC₅₀ of 558 nM, as opposed to 0.6 nM for MCF-7 cells. Taxotere® was also less active in the MDR-1 over-expressing with an IC₅₀ of 428 nM for NCl/ADR-RES cells versus 0.5 nM for MCF-7 cells.

TABLE 1
Cytotoxicity of 5-chloro-N-[2-(4-chloro-naphtalen-1-yloxy)-
5-trifluoromethyl-phenyl]-2-hydroxy-benzamide relative to
Vinblastine and Taxotere ® in MDR-1 overexpressing cells.

| | IC₅₀ (nM)* | | |
|---|---|---|---|
| | 5-chloro-N-[2-(4- | | |
| | chloro-naphtalen-1-yloxy)- | | |
| | 5-trifluoromethyl- | | |
| | phenyl]-2-hydroxy- | | |
| Cell Line | benzamide | Vinblastine | Taxotere ® |
| MCF-7 | 739 ± 38 | 0.6 ± 0.1 | 0.5 ± 0.2 |
| NCI/ADR-RES | 754 ± 38 | 558 ± 34 | 428 ± 27 |

*Values are Mean ± Standard deviation

### Conclusions

### 5-chloro-N-[2-(4-chloro-naphtalen-1-yloxy)-5-trifluoromethyl-phenyl]-2-hydroxy-benzamide is equally cytotoxic to MDR-1 over-expressing cell lines and normal cells.

### EXAMPLE 2

### Cytotoxicity of 5-chloro-N-[2-(4-chloro-naphtalen-1-yloxy)-5-trifluoromethyl-phenyl]-2-hydroxy-benzamide in multidrug resistant cells overexpressing MRP-1

Abstract

Objective: This study was conducted to investigate whether cells over-expressing the multidrug resistance associated protein 1 (MRP-1, ABCC1) are sensitive to the cytotoxic activity of 5-chloro-N-[2-(4-chloro-naphtalen-1-yloxy)-5-trifluoromethyl-phenyl]-2-hydroxy-benzamide in cell culture.

Methods: The tumor cells shown in Table 2 were plated at 5,000 cells/well in a 96 well tissue culture plate and grown for 24 hours at 37° C. The cells were then exposed to various concentrations of 5-chloro-N-[2-(4-chloro-naphtalen-1-yloxy)-5-trifluoromethyl-phenyl]-2-hydroxy-benzamide or Epirubicin for 72 hours and the inhibition of cell growth due to exposure to the drug was determined using the ATPLite assay. Data are expressed as the concentration that inhibited growth by 50%.

Results: The in vitro activity of 5-chloro-N-[2-(4-chloro-naphtalen-1-yloxy)-5-trifluoromethyl-phenyl]-2-hydroxy-benzamide and Epirubicin is compared in tumor cells and cells over-expressing MRP-1 in Table 2 below.

Conclusions: 5-chloro-N-[2-(4-chloro-naphtalen-1-yloxy)-5-trifluoromethyl-phenyl]-2-hydroxy-benzamide is equally active in tumor cells and cells over-expressing the MRP-1 transporter.

Introduction

Over-expression of the product of the ABCC1 gene encoding the multidrug resistance associated protein 1 confers resistance to a wide variety of agents including etoposide, methotrexate, cisplatins and anthracyclines. Cell lines with elevated expression of MRP-1 are less sensitive to the effect of these cytotoxic agents in vitro. MCF-7/VP cells, a MCF-7 derivative selected for resistance to etoposide, over-express the MRP-1 protein (1). The objective of this study was to determine whether MCF-7/VP cells are sensitive to the cytotoxic activity of 5-chloro-N-[2-(4-chloro-naphtalen-1-yloxy)-5-trifluoromethyl-phenyl]-2-hydroxy-benzamide in cell culture.

Experimental Methods

Test/Control Articles

A. 5-chloro-N-[2-(4-chloro-naphtalen-1-yloxy)-5-trifluoromethyl-phenyl]-2-hydroxy-benzamide (Myriad Pharmaceuticals, Salt Lake City, Utah)

B. Epirubicin (Calbiochem; San Diego, Calif.)

C. ATPLite® Assay Kit (PerkinElmer, Boston, Mass.)

Materials

Stock solutions of compounds dissolved in DMSO were used to prepare compound dilutions for activity determination. 5-chloro-N-[2-(4-chloro-naphtalen-1-yloxy)-5-trifluoromethyl-phenyl]-2-hydroxy-benzamide was tested at a final concentration of 100 μM, 33.3 μM, 11.1 μM, 3.7 μM, 1.23 μM, 0.4 μM and 0.13 μM for both cell lines. Epirubicin was tested at 10 μM, 3.3 μM, 1.1 μM, 0.37 μM, 0.12 μM, 0.04 μM and 0.013 μM for MCF-7 cells and at 100 μM, 33.3 μM, 11.1 μM, 3.7 μM, 1.23 μM, 0.4 μM and 0.13 μM for MCF-7/VP cells. The final concentration of DMSO in all cases was adjusted to 0.2% v/v.

Test System

A. MCF-7, a human breast cancer cell line was obtained from ATCC (Catalog number: HTB-22)

B. MCF-7/VP, a multi drug resistant cell line over-expressing MRP-1 was obtained from Dr. Erasmus Schneider, Wadsworth Center, NYS Dept. of Health, Albany, N.Y. 12237.

MCF-7 cells were cultured in RPMI-1640 supplemented with 10% fetal bovine serum, 2 mM Glutamax, 1 mM sodium pyruvate, 0.1 mM non-essential amino acids, 10 μg/ml recombinant human insulin and 10 mM Hepes. MCF-7/VP cells were cultured in DMEM high glucose supplemented with 10% fetal bovine serum, 2.5% horse serum, 4 mM Glutamax, 1 mM sodium pyruvate, 0.1 mM non-essential amino acids and 10 mM Hepes. Both cell lines were grown at 37° C. in a humidified 5% CO₂ atmosphere.

Experimental

For testing the effect of compounds on cell growth, exponentially growing cells were harvested by trypsinization and plated in fresh growth medium at 50,000 cells/ml, one hundred microlitres cell suspension per well, in a 96-well flat-bottomed microtiter plate (Corning, Costar 3595). Twenty-four hours later, culture medium was replaced with fresh growth medium and varying concentrations of test compound.

For both cell lines, cells were incubated with compound for three days and cellular viability was determined at the end of the incubation by measuring intracellular ATP with ATPLite assay system. All compounds were tested in triplicate.

Data Processing

Effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC₅₀). Data was analyzed by Prism software (GraphPad; San Diego, Calif.) by fitting it to a sigmoidal dose response curve. The IC₅₀ values obtained for individual data sets were combined to obtain a mean IC₅₀ and standard deviation of the mean.

Results

5-chloro-N-[2-(4-chloro-naphtalen-1-yloxy)-5-trifluoromethyl-phenyl]-2-hydroxy-benzamide showed similar cytotoxic activity against the normal and MRP-1 over-expressing cell line with IC₅₀'s of 592 and 800 nM for MCF-7 and MCF-7/VP cells respectively (Table 2).

Epirubicin, a substrate of the MRP-1 pump, was less active against the MRP-1 over-expressing cells MCF-7/VP with an IC₅₀ of 958 nM as opposed to 150 nM for MCF-7 cells.

TABLE 2
Cytotoxicity of 5-chloro-N-[2-(4-chloro-naphtalen-1-yloxy)-
5-trifluoromethyl-phenyl]-2-hydroxy-benzamide in
MRP-1 overexpressing cells.

IC50 (nM)*

| | 5-chloro-N-[2-(4-chloro- | |
|---|---|---|
| | naphtalen-1-yloxy)-5- | |
| | trifluoromethyl-phenyl]-2- | |
| Cell Line | hydroxy-benzamide | Epirubicin |
| MCF-7 | 592 ± 20 | 150 ± 43 |
| MCF-7/VP | 800 ± 116 | 958 ± 114 |

*Values are Mean ± Standard deviation

### Conclusions

5-chloro-N-[2-(4-chloro-naphtalen-1-yloxy)-5-trifluoromethyl-phenyl]-2-hydroxy-benzamide is equally cytotoxic to MRP-1 over-expressing cell lines and normal cells.

### REFERENCES

(1) Schneider E, Horton J K, Yang C H, Nakagawa M, Cowan K H. Multidrug resistance-associated protein gene overexpression and reduced drug sensitivity of topoisomerase II in a human breast carcinoma MCF7 cell line selected for etoposide resistance. Cancer Res 54:152-8 (1994).

### EXAMPLE 3

### Cytotoxicity of 5-chloro-N-[2-(4-chloro-naphtalen-1-yloxy)-5-trifluoromethyl-phenyl]-2-hydroxy-benzamide in multidrug resistant cells overexpressing BCRP

Abstract

Objective: This study was conducted to investigate whether cells over-expressing the breast cancer resistance protein (BCRP/MXR/ABCG2) are sensitive to the cytotoxic activity of 5-chloro-N-[2-(4-chloro-naphtalen-1-yloxy)-5-trifluoromethyl-phenyl]-2-hydroxy-benzamide in cell culture.

Methods: The tumor cells were plated at 5,000 cells/well in a 96 well tissue culture plate and grown for 24 hours at 37° C. The cells were then exposed to various concentrations of 5-chloro-N-[2-(4-chloro-naphtalen-1-yloxy)-5-trifluoromethyl-phenyl]-2-hydroxy-benzamide, Camptothecin or Epirubicin for 72 hours and the inhibition of cell growth due to exposure to the drug was determined using the ATPLite assay. Data are expressed as the concentration that inhibited growth by 50%.

Results: The in vitro activity of 5-chloro-N-[2-(4-chloro-naphtalen-1-yloxy)-5-trifluoromethyl-phenyl]-2-hydroxy-benzamide, Camptothecin and Epirubicin is compared in tumor cells and BCRP over-expressing tumor cells in Table 3 below.

Conclusions: 5-chloro-N-[2-(4-chloro-naphtalen-1-yloxy)-5-trifluoromethyl-phenyl]-2-hydroxy-benzamide is equally active in tumor cells and cells over-expressing the BCRP transporter.

Introduction

Over-expression of the product of the ABCG2 gene encoding the BCRP (MXR) protein confers resistance to a wide variety of agents including mitoxantrone, camptothecins and antharacyclines. Cell lines with elevated expression of BCRP are less sensitive to the effect of these cytotoxic agents in vitro. MCF-7/MX cells, a MCF-7 derivative selected for resistance to mitoxantrone, over-express the BCRP protein (1). The objective of this study was to determine whether MCF-7/MX cells are sensitive to the cytotoxic activity of 5-chloro-N-[2-(4-chloro-naphtalen-1-yloxy)-5-trifluoromethyl-phenyl]-2-hydroxy-benzamide in cell culture.

Experimental Methods

Test/Control Articles

A. 5-chloro-N-[2-(4-chloro-naphtalen-1-yloxy)-5-trifluoromethyl-phenyl]-2-hydroxy-benzamide (Myriad Pharmaceuticals, Salt Lake City, Utah)

B. Epirubicin (Calbiochem; San Diego, Calif.)

C. CPT-11 (Camptosar®; Pharmacia Upjohn, Kalamazoo, Mich.)

D. ATPLite® Assay Kit (PerkinElmer, Boston, Mass.)

Materials

Stock solutions of compounds dissolved in DMSO were used to prepare compound dilutions for activity determination. 5-chloro-N-[2-(4-chloro-naphtalen-1-yloxy)-5-trifluoromethyl-phenyl]-2-hydroxy-benzamide was tested at a final concentration of 100 μM, 33.3 μM, 11.1 μM, 3.7 μM, 1.23 μM, 0.4 μM and 0.13 μM for both cell lines. CPT-11 was tested at 1 μM, 0.33 μM, 0.1 μM, 0.04 μM, 0.012 μM, 0.004 μM and 0.001 μM in MCF-7 cells and at 10 μM, 3.3 μM, 1.1 μM, 0.37 μM, 0.12 μM, 0.04 μM and 0.013 μM in MCF-7/MX cells. Epirubicin was tested at 10 μM, 3.3 μM, 1.1 μM, 0.37 μM, 0.12 μM, 0.04 μM and 0.013 μM for MCF-7 cells and at 100 μM, 33.3 μM, 11.1 μM, 3.7 μM, 1.23 μM, 0.4 μM and 0.13 μM for MCF-7/MX cells. The final concentration of DMSO in all cases was adjusted to 0.2% v/v.

Test System

A. MCF-7, a human breast cancer cell line was obtained from ATCC (Catalog number: HTB-22)

B. MCF-7/MX, a multi drug resistant cell line over-expressing BCRP was obtained from Dr. K. Cowan, University of Nebraska Medical Center, 986805 Nebraska Medical Center, Omaha, Nebr. 68198.

Both cell lines were cultured in RPMI-1640 supplemented with 10% fetal bovine serum, 2 mM Glutamax, 1 mM sodium pyruvate, 0.1 mM non-essential amino acids, 10 μg/ml recombinant human insulin and 10 mM Hepes. All cell lines were grown at 37° C. in a humidified 5% CO₂ atmosphere.

Experimental

For testing the effect of compounds on cell growth, exponentially growing cells were harvested by trypsinization and plated in fresh growth medium at 50,000 cells/ml, one hundred microlitres cell suspension per well, in a 96-well flat-bottomed microtiter plate (Corning, Costar 3595). Twenty-four hours later, culture medium was replaced with fresh growth medium and varying concentrations of test compound.

For both cell lines, cells were incubated with compound for three days and cellular viability was determined at the end of the incubation by measuring intracellular ATP with ATPLite assay system. All compounds were tested in triplicate.

Data Processing

Effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC₅₀). Data was analyzed by Prism software (GraphPad; San Diego, Calif.) by fitting it to a sigmoidal dose response curve. The IC₅₀ values obtained for individual data sets were combined to obtain a mean IC₅₀ and standard deviation of the mean.

Results

5-chloro-N-[2-(4-chloro-naphtalen-1-yloxy)-5-trifluoromethyl-phenyl]-2-hydroxy-benzamide showed a two-fold difference in cytotoxic activity against the normal and BCRP over-expressing cell line with IC₅₀'s of 739 and 1480 nM for MCF-7 and MCF-7/MX cells respectively (Table 3).

CPT-11 and Epirubicin, which are substrates of the BCRP pump, were one-eighth and one-seventh less active against the BCRP over-expressing cells MCF-7/MX with IC₅₀'s of 174 nM and 1115 nM respectively as opposed to 21.9 nM and 150 nM for MCF-7 cells.

TABLE 3
Cytotoxicity of 5-chloro-N-[2-(4-chloro-naphtalen-1-yloxy)-5-
trifluoromethyl-phenyl]-2-hydroxy-benzamide in
BCRP overexpressing cells.

| | IC₅₀ (nM)* | | |
|---|---|---|---|
| | 5-chloro-N-[2-(4- | | |
| | chloro-naphtalen-1- | | |
| | yloxy)-5- | | |
| | trifluoromethyl- | | |
| | phenyl]-2-hydroxy- | | |
| Cell Line | benzamide | Camptothecin ® | Epirubicin |

| | | | |
|---|---|---|---|
| MCF-7 | 739 ± 38 | 21.9 ± 4.9 | 150 ± 43 |
| MCF-7/MX | 1480 ± 190 | 174 ± 7 | 1115 ± 213 |

*Values are Mean ± Standard deviation

Conclusions

A BCRP over-expressing cell line is only twofold more resistant to 5-chloro-N-[2-(4-chloro-naphtalen-1-yloxy)-5-trifluoromethyl-phenyl]-2-hydroxy-benzamide than normal cells.

### REFERENCES

(1) Nakagawa M, Schneider E, Dixon K H, Horton J, Kelley K, Morrow C and Cowan K H. Reduced intracellular drug accumulation in the absence of P-glycoprotein (mdrl) overexpression in mitoxantrone-resistant human MCF-7 breast cancer cells. Cancer Res 52:6175-81 (1992).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method of treating refractory cancer comprising treating a patient having refractory cancer with a therapeutically effective amount of 5-chloro-N-[2-(4-chloro-naphtalen-1-yloxy)-5-trifluoromethyl-phenyl]-2-hydroxy-benzamide, or a pharmaceutical salt thereof.

2. The method of claim 1, wherein said refractory cancer is resistant to, or has recurred following, treatment with one or more antineoplastic agents.

3. A method of treating refractory cancers comprising
identifying a patient with a refractory cancer and
administering a therapeutically effective amount of 5-chloro-N-[2-(4-chloro-naphtalen-1-yloxy)-5-trifluoromethyl-phenyl]-2-hydroxy-benzamide, or a pharmaceutical salt thereof, to the patient.

4. The method of claim 3, wherein the refractory cancer failed to respond to an initial treatment regimen with one or more antineoplastic agents other than 5-chloro-N-[2-(4-chloro-naphtalen-1-yloxy)-5-trifluoromethyl-phenyl]-2-hydroxy-benzamide, or relapsed following an initially favorable response to such an initial treatment regimen.

5. The method of claim 4, wherein the failed response to an initial treatment regimen or the relapse is revealed by an increase in original tumor size, or the presence of new lesions.

6. The method of claim 5, wherein the increase in the original tumor size is at least about a 10% increase in size as measured along the longest axis.

7. The method of claim 5, wherein the increase in the original tumor size is at least about a 10% increase in volume.

8. The method of claim 4, wherein the failed response to an initial treatment regimen, or the relapse, is revealed by an increase in blood borne cancer cells.

9. The method of claim 4, wherein the patient has a multidrug resistant (MDR) cancer.

10. The method of claim 9, wherein the MDR cancer is confirmed by a diagnostic procedure that measures the expression of a gene encoding an ATP-binding cassette (ABC) transporter protein in cancer cells isolated from said patient.

11. The method of claim 10, wherein said diagnostic procedure is selected from:
(a) quantitative RT-PCR of ABC transporter protein-encoding mRNAs,
(b) expression profiling of ABC transporter protein-encoding nucleic acids with microarrays,
(c) ELISAs utilizing antibodies that specifically bind with ABC transporter proteins, or
(d) flow cytometry utilizing antibodies that specifically bind ABC transporter proteins.

12. The method of claim 4, wherein said initial treatment regimen comprised treatment with one or more of the following: alkylating agents, antimitotic agents, tubulin inhibitors, topoisomerase I inhibitors, topoisomerase II inhibitors, RNA/DNA antimetabolites, DNA antimetabolites, EGFR inhibitors, angiogenesis inhibitors, proteosome inhibitors, or combinations thereof.

13. The method of claim 4, wherein said initial treatment regimen comprised treatment with one or more of the following: anthracyclines, Vinca-alkaloids, taxanes, metal complexes, or combinations thereof.

14. The method of claim 4, wherein said initial treatment regimen comprised treatment with one or more of the following: actinomycin-D, bleomycin, bisantrene, aclarubicin, doxorubicin, daunorubicin, epirubicin, idarubicin, docetaxel, paclitaxel, etoposide, teniposide, topotecan, mitoxantrone, vinblastine, vincristine, vinorelbine, homoharringtonine, cisplatin, chlorambucil, melphalan, cyclophosamide, ifosfamide, mitoguazone, elliptinium, fludarabine, octreotide, retinoic acid, tamoxifen, Gleevec® (imatinib mesylate), Alanosine, SN-38, or combinations thereof.

15. The method of claim 4, wherein the cancer to be treated is characterized by the presence of solid tumors.

16. The method of claim 15, wherein the cancer to be treated is a metastatic cancer.

17. The method of claim 15, wherein the cancer to be treated is a cancer of the skin, colon, rectum, esophagus, thyroid, liver, pancreas, kidney, bladder, lung, brain, breast, ovary, testicle or prostate.

18. A method of treating MDR cancer comprising treating a patient having MDR cancer with a therapeutically effective amount of 5-chloro-N-[2-(4-chloro-naphtalen-1-yloxy)-5-trifluoromethyl-phenyl]-2-hydroxy-benzamide, or a pharmaceutical salt thereof.

19. A method of treating an MDR cancer in a patient in need of such treatment comprising
identifying a patient previously treated with a antineoplastic agent whose cancer was refractory to, or relapsed from, the previous antineoplastic treatment, and
administering a therapeutic amount of 5-chloro-N-[2-(4-chloro-naphtalen-1-yloxy)-5-trifluoromethyl-phenyl]-2-hydroxy-benzamide, or a pharmaceutical salt thereof.

20. The method of claim 19, wherein the previous antineoplastic treatment comprised administration of or more of the following: alkylating agents, antimitotic agents, tubulin inhibitors, topoisomerase I inhibitors, topoisomerase II inhibitors, RNA/DNA antimetabolites, DNA antimetabolites, EGFR inhibitors, angiogenesis inhibitors, proteosome inhibitors, or combinations thereof.

21. The method of claim 19, wherein the previous antineoplastic treatment comprised administration of or more of the following: anthracyclines, Vinca-alkaloids, taxanes, metal complexes, or combinations thereof.

22. The method of claim 19, wherein the previous antineoplastic treatment comprised administration of or more of the following: actinomycin-D, bleomycin, bisantrene, aclarubicin, doxorubicin, daunorubicin, epirubicin, idarubicin, docetaxel, paclitaxel, etoposide, teniposide, topotecan, mitoxantrone, vinblastine, vincristine, vinorelbine, homoharringtonine, cisplatin, chlorambucil, melphalan, cyclophosamide, ifosfamide, mitoguazone, elliptinium, fludarabine, octreotide, retinoic acid, tamoxifen, Gleevec® (imatinib mesylate), Alanosine, SN-38, or combinations thereof.

23. The method of claim 19, wherein the multiple drug-resistant cancer is a cancer of the skin, colon, rectum, esophagus, thyroid, liver, pancreas, kidney, bladder, lung, brain, breast, ovary, testicle or prostate.

24. The method of claim 19, wherein the patient in need of such treatment is a patient previously treated with more than one antineoplastic agents, and whose cancer was refractory to, or relapsed from, any of the previous treatments.

25. A method of killing MDR cancer cells comprising contacting the MDR cancer cells with 5-chloro-N-[2-(4-chloro-naphtalen-1-yloxy)-5-trifluoromethyl-phenyl]-2-hydroxy-benzamide, or a pharmaceutical salt thereof.

26. A method of treating refractory cancers in human patients in need of such treatment comprising the steps of:
(a) identifying a patient having a refractory cancer,
(b) administering to said patient a therapeutically effective amount of 5-chloro-N-[2-(4-chloro-naphtalen-1-yloxy)-5-trifluoromethyl-phenyl]-2-hydroxy-benzamide, or a pharmaceutical salt thereof, and
(c) assessing the progression of the disease.

27. The method of claim 26 wherein the refractory cancer is identified by a diagnostic procedure that measures the amount of ABC transporter protein gene expression in cancer cells isolated from the patient.

28. Use of 5-chloro-N-[2-(4-chloro-naphtalen-1-yloxy)-5-trifluoromethyl-phenyl]-2-hydroxy-benzamide, or a pharmaceutical salt thereof, in the manufacture of a medicament for the treatment of refractory cancers in patients in need of such treatment.

29. The use of claim 28, wherein said refractory cancer is resistant to, or has recurred following treatment with one or more antineoplastic agents.

30. The use of claim 28, wherein said refractory cancer is characterized by the presence of solid tumors.

31. The use of claim 28, wherein said refractory cancer is a metastatic cancer.

32. The use of claim 28, wherein said refractory cancer is a cancer of the skin, colon, rectum, esophagus, thyroid, liver, pancreas, kidney, bladder, lung, brain, breast, ovary, testicle or prostate.

33. The use of claim 28, wherein said refractory cancer is a cancer that is refractory to previous treatment with one or more of the following antineoplastic agents: alkylating agents, antimitotic agents, tubulin inhibitors, topoisomerase I inhibitors, topoisomerase II inhibitors, RNA/DNA antimetabolites, DNA antimetabolites, EGFR inhibitors, angiogenesis inhibitors, proteosome inhibitors, or combinations thereof.

34. The use of claim 28, wherein said refractory cancer is a cancer that is refractory to previous treatment with one or more of the following antineoplastic agents: anthracyclines, Vinca-alkaloids, taxanes, metal complexes, or combinations thereof.

35. The use of claim 28, wherein said refractory cancer is a cancer that is refractory to previous treatment with one or more of the following antineoplastic agents: actinomycin-D, bleomycin, bisantrene, aclarubicin, doxorubicin, daunorubicin, epirubicin, idarubicin, docetaxel, paclitaxel, etoposide, teniposide, topotecan, mitoxantrone, vinblastine, vincristine, vinorelbine, homoharringtonine, cisplatin, chlorambucil, melphalan, cyclophosamide, ifosfamide, mitoguazone, elliptinium, fludarabine, octreotide, retinoic acid, tamoxifen, Gleevec® (imatinib mesylate), Alanosine, SN-38, or combinations thereof.
